(19) United States
(12) Patent Application Publication
(10) Pub. No.: US 2011/0118834 A1
(43) Pub. Date: May 19, 2011

(54) FLUIDIC INTRAOCULAR LENS SYSTEMS AND METHODS

(76) Inventors: Yuhwa Lo, San Diego, CA (US); Wen Qiao, La Jolla, CA (US); David Schanzlin, La Jolla, CA (US); Gary L. Shields, Rancho Santa Fe, CA (US); Robert S. Vasko, San Diego, CA (US); Jeff Vasko, San Diego, CA (US)

(21) Appl. No.: 12/874,150

(22) Filed: Sep. 1, 2010

Related U.S. Application Data
(63) Continuation-in-part of application No. 12/256,961, filed on Oct. 23, 2008.
Application No. 12/256,961 is a continuation-in-part of application No. 11/683,141, filed on Mar. 7, 2007, now Pat. No. 7,453,646.
Application No. 11/683,141 is a continuation-in-part of application No. 10/599,486, filed on Mar. 8, 2007, now Pat. No. 7,675,686.
(60) Provisional application No. 60/558,293, filed on Mar. 31, 2004.

(51) Int. Cl. A61F 2/16; G02C 7/02; A61B 3/10

(52) U.S. Cl. 623/6.13; 351/177; 351/246

(57) ABSTRACT

The present invention relates to a fluidic intraocular lens inserted into a capsular bag of an eye to replace a crystalline lens. The fluidic intraocular lens may be shaped by elastic membranes bonded to a support ring. The space inside the device may be filled with optically transparent fluid or gel having an index of refraction greater than the index of vitreous humor. The device may be such designed so that the focusing power of the lens can be changed by the deformation of the capsular bag, which may be subsequently controlled by a ciliary muscle.

### CROSS-REFERENCE TO RELATED APPLICATIONS

This application is a continuation-in-part of U.S. patent application Ser. No. 12/256,961 entitled “Fluidic Adaptive Lens Systems and Methods” filed on Oct. 23, 2008, which is a continuation-in-part of U.S. patent application Ser. No. 11/683,141 entitled “Fluidic Adaptive Lens Systems and Methods” filed on Mar. 7, 2007, which is a continuation-in-part of U.S. patent application Ser. No. 10/599,486, which is the US. national phase patent application of International Application No. PCTUS2005010948 entitled “Fluidic Adaptive Lens” filed on Mar. 31, 2005, and which claims priority to U.S. provisional application No. 60/558,293 entitled “Fluidic Adaptive Lens” filed on Mar. 31, 2004, each of which is hereby incorporated by reference herein.

### BACKGROUND OF THE INVENTION

FIG. 1 shows a human eye 2 that comprises a cornea 7, an iris 3, and a crystalline lens 9 that form an optical system that bends light and creates an image on a retina 8. The crystalline lens 9 not only contributes approximately one-third of the total optical power for image formation, but also adjusts the focal length so that clear images of objects from different distances can be formed on the retina 8. The crystal line lens 9 may be suspended in the vitreous humor and is mechanically coupled to the ciliary muscle 4 via the suspensory ligament and fibrous zonules 5 around the equator of the lens. In the natural state where the eye may be focused on far objects, there exists a built-in tension on the zonules 5 and such tension makes the lens stretched to a longer and thinner shape than it may be without the radial tension. During accommodation, the ciliary muscle 4 contracts and the tension in the zonules 5 decreases. The decreased tension in the zonules 5 reduces the equatorial diameter of the capsular bag 6 and produces a shorter, thicker and more curved crystalline lens 9 with greater optical power to focus on nearer objects. The change of equatorial diameter of the bag during accommodation varies from 1 mm for a 21-year old lens to about 0.5 mm for a 41-year old lens and to around 0.3 mm at an older age, indicating the gradual loss of the accommodation ability of eyes.

According to the World Health Organization (WHO), the population of blindness was 37 million in 2002. The leading reason for blindness may be an age-related cataract, causing the blindness of 18 million people. Responsible for 48% of blindness, the cataract may be a main cause impacting human health. The cataract may be developed when a crystalline lens becomes cloudy and finally blocks the transmission of light. Fortunately, cataract patients can be cured by appropriate cataract operation, during which the cloudy lens may be removed from capsular bag and a synthetic lens, also known as intraocular lens (IOL), may be inserted. An AOL can change its optical focusing power so that objects from near and far distances may be seen. Such a device behaves in a similar manner as a natural crystalline lens and eliminates many performance compromises with multifocal IOLs. With accommodating IOLs of sufficient lens power and tuning range, the clarity and flexibility of vision can be restored for daily routines such as driving, reading, entertaining, etc. without additional correction lenses.

The optical tuning capacity of an IOL may be defined as the amount of change in optical power measured in Dioptres (D). A healthy young eye can typically achieve an 8 D accommodation range, which means that the person can clearly see objects from a far distance to as close as 12.5 cm. The latter number may be obtained from the simple relation 12.5=100/8. Although a tuning range of 8 D, in principle, restores the vision of a cataract patient, an ideal accommodating IOL should have larger accommodating range than a natural lens in the same condition. With aging, the capsular bag may be thickened and becomes more rigid so the extent of shape change may be reduced under the same amount of muscle force. Moreover, both the zonules, connecting tissues between the capsular bag and the ciliary muscle, and the capsular bag become brittle after the surgery. Only when the IOL can achieve an accommodation range comparable to the crystalline lens but at a smaller change of the shape of the capsular bag and at a smaller magnitude of muscle force, the vision of aged patients can be sufficiently improved.

Because of the strong demand, many efforts have been devoted to developing accommodating IOLs over the past few decades.

U.S. Pat. No. 6,638,306 illustrates a single-piece accommodating IOL having T-shaped haptics. After being implanted in the capsular bag, the optics can move along the visual axis back and forth in accordance with the applied force. The change of the optical power may be obtained by varying the distance between the IOL and the retina. The accommodation range depends on the travel distance of the lens within the capsular bag, typically 2-3 mm at maximum. As a result, the tuning power of the IOL may be theoretically limited to 1.9 D.

U.S. Pat. No. 7,041,134 discloses another camera-like accommodating IOL consisting of two lenses connected by a bridge structure attached to the haptics. The device may be designed to change the spacing between the two lenses by the tension of the capsular bag. The larger the travel distance of the lenses, the higher may be the tuning range. Compared with the single optic-shifting IOL elucidated in U.S. Pat. No. 6,638,306, the design augments the accommodation range to 2.5 D, limited again by the constrained spacing of the capsular bag.

PCT International Application No. PCT/IL02/00693, of PCT International publication No. WO 03/015669, discloses an accommodating IOL including a haptics structure for implantation in the posterior chamber rather than in the capsular bag of an eye. The device has a deformable lens that can be pressed by the axial force of the capsular bag. The change of the force alters the focal power of the IOL.

U.S. Pat. No. 7,261,737 describes an accommodating IOL with a flexible fluid-filled piston actuator, connected to the haptics through fluidic channels. When forces are applied to the haptics, fluid moves into the central actuator, pushing the inter surface of the lens and resulting in a more curved lens of increasing optical power. Because the piston structure may be in the optical path, the material needs to be index matched to its surroundings, imposing strict limits to the choices of the piston material and the lens fluid.

### DESCRIPTION OF FIGURES

FIG. 1 shows across section of human eye 2, including an Iris 3, a ciliary muscle 4, zonules 5, a capsular bag 6, a cornea 7, a retina 8, a crystalline lens 9;

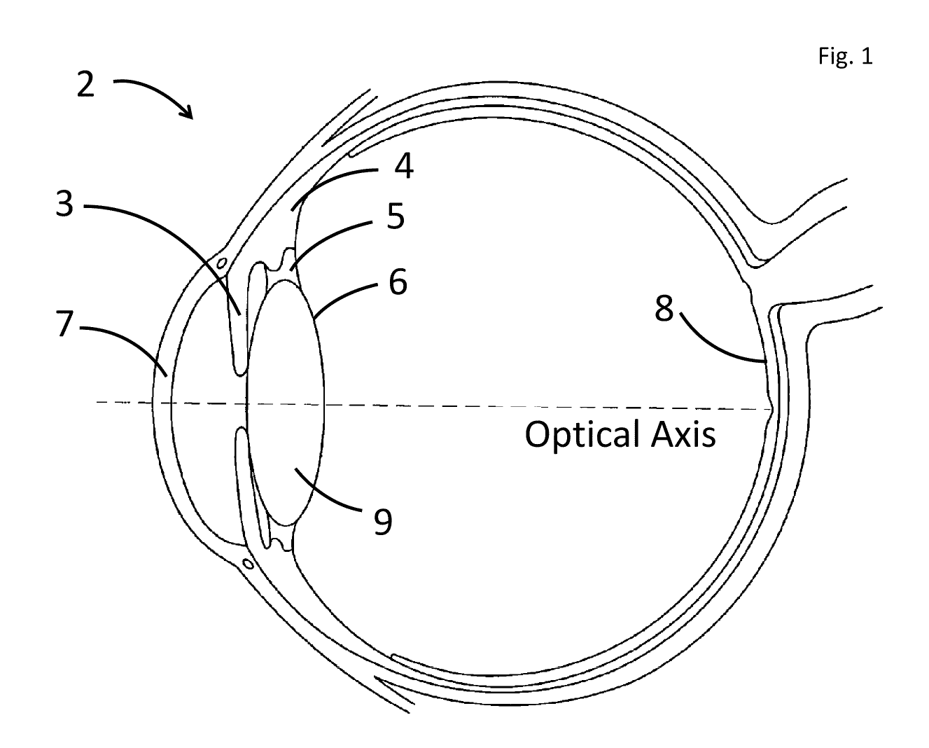

FIG. 2 shows side views of the a fluidic intraocular lens (IOL), including an elastic membrane 21 forming the anterior surface of the IOL, an elastic membrane 22 forming the posterior surface of the IOL, a supporting ring 23 to which the membranes are attached, lens chamber 24, a peripheral reservoir 25, an optical fluid 26, through holes 27 to allow the flow of optical fluid between the lens chamber 24 and the peripheral reservoir 25, and also shows that a compressive force to the flexible wall of peripheral reservoir causes an increase in the curvature of the lens membranes;

FIG. 3 shows a different view of the fluidic IOL of FIG. 2, including a channel 28 for fluid fill, where one opening of the channel 28 may be on the surface of the ring 23 in contact with a membrane 29, and another opening of the channel 28 may be on the side wall of the ring 23 in contact with a lens chamber 30, where optical fluid may be injected via the channel 28 by piercing a hole through the membrane 29, and where the channel 28 may be sealed or plugged after fluidic fill;

FIG. 4A shows a fluidic IOL 40 in accordance with the present invention;

FIG. 4B shows an internal view of fluidic IOL 40 of FIG. 4A;

FIG. 5A shows a fluidic IOL 50 in accordance with the present invention;

FIG. 5B shows a cross sectional view of the fluidic IOL 50 of FIG. 5A; and

FIG. 6 illustrates a method of fabrication of an accommodating fluidic IOL, showing a forming 610 of an optically transparent elastic membranes on a silicon wafer by spin coating, forming 620 of a ring structure out of flexible material with holes for fluid transport and channel(s) for fluid fill, a joining 630 of top and bottom membranes with the ring, where the peripheral of the two membranes are bonded and each membrane may be bonded to each side of the surface of the ring, and a formation 640 of a fluidic IOL after fluid fill and seal of the fill channel.

### DESCRIPTION OF THE INVENTION

The present invention addresses the design and performance of accommodating fluidic IOLs. The inventive IOL may be a disk-like fluidic lens implantable to a capsular bag of an eye. The device can efficiently couple the limited amount of deformation of capsular bag during accommodation to the shape changes of the lens under a modest force of the ciliary muscle. The fluidic IOL may comprise two membranes, optical fluid or gel, and a support ring that defines the reservoir of the fluidic IOL and separates a fluidic lens chamber from the reservoir. The ring may have multiple holes on a side wall to allow fluid exchange between the lens chamber and the reservoir. By compressing the flexible reservoir of the fluidic IOL radially, the fluid/gel in the reservoir can move into the lens chamber, and the curvature of the lens increases for higher optical power.

In a cataract surgery, a crystalline lens may be removed from a capsular bag and a fluidic IOL may be inserted in the vacant space. The fluidic IOL may be made of bendable material to work with a fluidic IOL dispensing device. The lens may be introduced in a folded form through an incision on the capsular bag. After being placed inside the capsular bag, the fluidic IOL may be configured to unfold or unbend. In its free form outside the eye, the outer diameter of the fluidic IOL may be slightly larger than the inner equatorial diameter of the capsular bag. After insertion, the radial tension exerted by the capsular bag to the fluidic IOL keeps the IOL in position. The pressure to the peripheral reservoir pushes the fluid into the lens chamber of the fluidic IOL to achieve proper optical power to form an image of far objects in conjunction with the optical power from the cornea.

As a ciliary muscle contracts, zonules connecting the ciliary muscle and the capsular bag lose the tension that keeps the capsular bag stretched. Consequently, the capsular bag returns to a thicker shape with a reduced equatorial diameter. The fluidic IOL inside the capsular bag may be subsequently squeezed into a thicker shape with a stronger optical power, enabling the eye to focus on nearer objects.

Numerical simulations suggest that the maximum force of the ciliary muscle force may be around 8 grams (0.08 Newtons). Under such force, the diameter of the capsular bag could be reduced by 1 mm for young eyes, 0.5 mm for 40-years-old eyes, and around 0.3 mm for more aged eyes. As people age, the wall of the capsular bag thickens. Therefore, the same amount of muscle force may not produce the same amount of change in the shape of the capsular bag. Although the structural and mechanical changes of eye for different patients may vary widely, it may be always desirable to design the fluidic IOL to be suitable for a majority of patients under varying circumstances. The majority of patents having cataract surgery may be in their middle or senior age, and the accommodating fluidic IOL may be designed to achieve sufficient optical tuning power under a contraction of capsule diameter no greater than 0.3 mm and a compressive force no greater than 6 grams. In other words, the device would need to deliver more tuning power than a crystalline lens in a healthy young human eye under a given muscle force and capsular shape change to compensate for the effect of aging. To meet this challenge, one effective approach may be to fill the lens chamber with higher index fluid than the crystalline lens, which has an average index of around 1.396 for an index gradient from 1.386 to 1.406. The shape of the lens, the index of refraction of the fluid, and its color dispersion characteristics may need to be modeled to assure not only sufficient accommodation, but also high image resolution may be achieved.

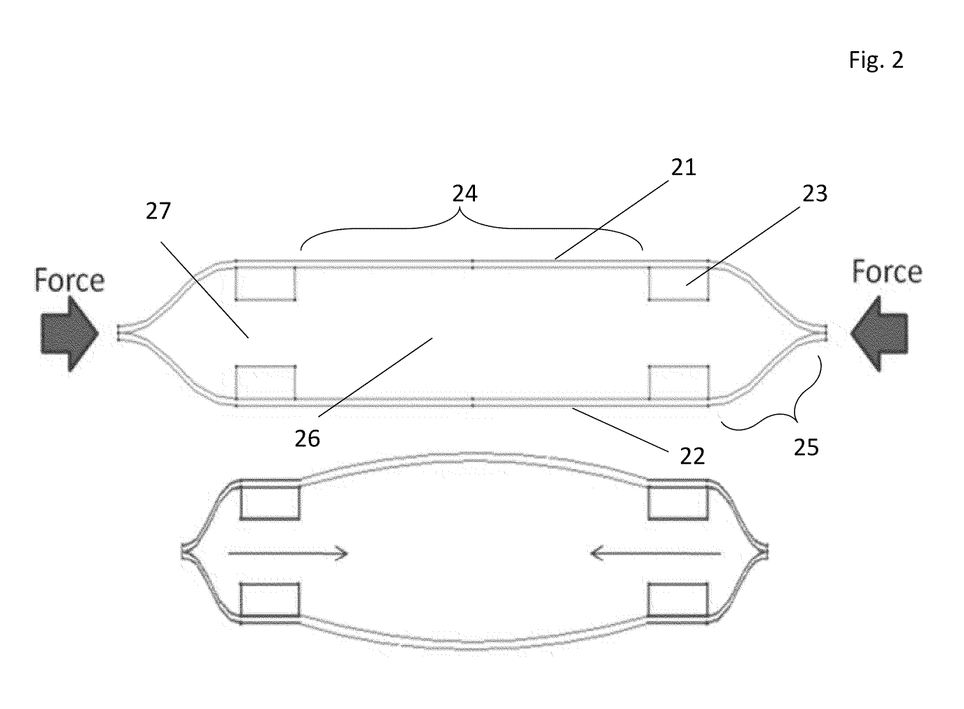

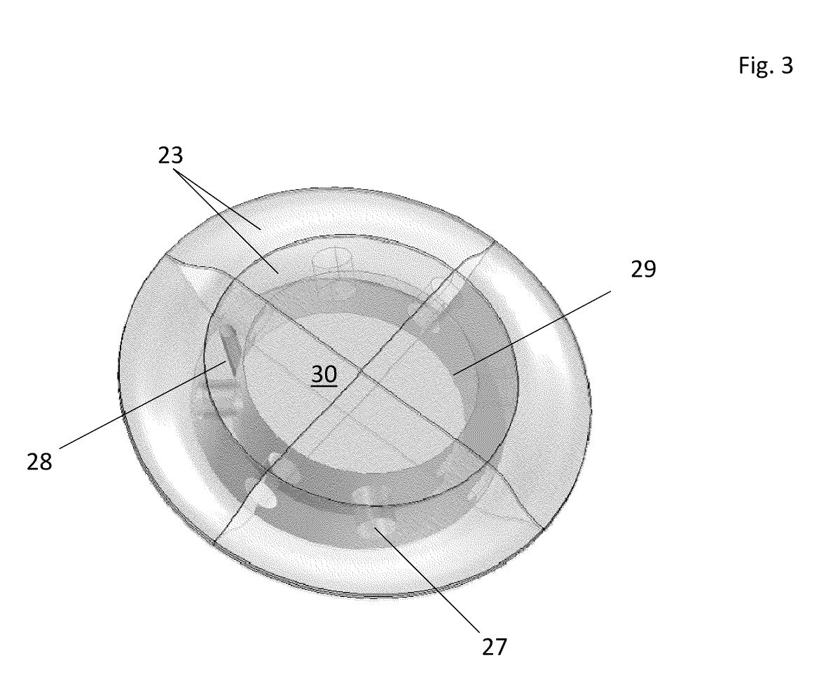

A fluidic IOL design that meets the above objectives may be illustrated in FIG. 2. The accommodating fluidic IOL consists of a tunable lens at the center and a fluid reservoir next to the lens region. The tunable lens may be made of two elastic membranes bonded to the surfaces of a support ring to form a fluid containing chamber. The fluid in the lens chamber may be the main optical medium and has a different index of refraction from the background, which has an index of about 1.33. When an amount of fluid in the lens chamber increases, the pressure in the chamber builds up and the curvatures of the two elastic membranes increase accordingly, producing a stronger optical power. The change of the anterior and posterior lens surfaces could be different, depending on the thickness and composition of the membranes. The fluid reservoir next to the lens may be also enclosed by elastic membranes held together through bonding. Fluid exchange can occur between the reservoir and the lens chamber through channels. One way to create such channels may be to create through holes on the sidewall of the ring, as shown in see FIG. 3. Because the two chambers may be connected without valves, the pressure in both chambers remains equal.

The typical diameter of the capsular bag may be 9 to 10 mm for most people. If the outer diameter (e.g. 11 mm) of the fluidic IOL may be slightly greater than the diameter of capsular bag, the fluidic IOL may be compressed after insertion to cause an increase in the optical power of the lens. Force may be applied to the lens through the contact between the reservoir and the inner wall of capsular bag. To inject the optical fluid into the lens chamber and reservoir during device fabrication, a channel may be formed between the top surface and the side wall of the ring. The angle and the position of the channel may be chosen to minimize its influence on the circularity of the support ring and its impact on the integrity of the structure. A sharp needle can be used to pierce a hole through the covering membrane for fluid injection. After fluid fill, the injection channel may be plugged or sealed.

The membrane in accordance with the present invention may be thin and stretchable, and has low modulus (e.g. elastomer). In the lens area, the membrane may also be optically transparent and smooth. A root-mean-square (RMS) surface roughness of less than 3 nm may be desired. The thickness of the membrane may be typically between 10 μm and 500 μm. In addition, the membrane may be biocompatible and impermeable to the optical fluid in the IOL.

The support ring can define the optical aperture and provide mechanical support for the lens. The support ring may have channels (e.g., 8 channels) through the wall to connect the outer reservoir and the lens chamber. The number and the dimension of the channels can be adjusted to minimize the resistance of the fluidic flow to acquire a short response time. One distinctive advantage of the invention compared to other IOLs may be that the ring assures the circularity and concentricity of the lens even if the patient's capsular bag does not have an ideal circular and symmetric geometry. After an incision may be created on the surface of the capsular bag and the cataract may be removed, there may be no guarantee that the pressure applied to the inserted IOL will be uniform along the equator of the capsular bag, thus the outer reservoir may be deformed irregularly. However, the inventive ring, although bendable, may be significantly more rigid than the membranes, and the shape of the lens will therefore always remain circular and centered. The ring may be configured to mechanically decouple the deformation of the reservoir from the lens membrane. As a result, the lens will not suffer from astigmatism and distortion caused by the non-uniform compression of the capsular bag. In addition, since two lens surfaces (anterior and posterior surfaces) share the same lens chamber and may be formed on either side of the same ring, the optical axes of the two lens surfaces may be aligned.

The refractive index of the fluid can be different from the index of vitreous humor to obtain the lens function. The index of the fluid may be typically in the range of 1.33-1.67. Higher refractive index may be preferred because it offers a wider range of tuning under given amounts of force and displacement. For the same optical power, the fluidic lens of higher index fluid may also have a lower curvature, resulting in a smaller amount of geometric aberration including spherical aberration, coma, field curvature, and astigmatism. However, higher index fluid often has stronger color dispersion and therefore suffers from a stronger effect of chromatic dispersion. The amount of chromatic dispersion in the visible spectrum may be often characterized by an Abbe number. A low Abbe number represents stronger color dispersion. Therefore, in the choice of the index of refraction of fluid, a tradeoff may be made between the value of the index of refraction and its Abbe number. Another point to consider, in choosing the optical fluid, may be the image field. Unlike cameras and most human-made imaging devices, high image acuity (about 1 arc minute) only occurs within a small angle of field no greater than 10 degrees. Images outside the fovea may be of low resolution, and treated as peripheral vision. Therefore, although unacceptable to human made imaging systems, it may be all right to choose the fluid and IOL structure to deliver high image resolution in the fovea with a field of view of around 10 degrees and show lower quality off-axis images for peripheral vision. Since both geometric aberration and chromatic dispersion worsen rapidly as the image field moves away from the optical axis, the relaxed image requirement for peripheral vision presents a different parameter optimization space than conventional imaging optics. Taking all the above factors into consideration and through a survey of optical fluid suitable for the IOL application, we have found that an optimum index value of the fluid may fall in the range of 1.40-1.58.

Although the membrane may be impermeable to the optical fluid, the optical fluid may need to be biocompatible in case of tearing of the membrane. To reduce possible dislocation of the fluidic IOL, and to minimize the distortion of lens membrane due to the effect of gravity, it may be also desirable to choose fluid having a similar density to the surrounding medium.

Different patients have different eye anatomy, characterized by the power of cornea and the distance between the cornea and the retina. After cataract removal, the fluidic IOL may need to produce the correct amount of power for patients to clearly see far objects when the ciliary muscle may be in its relaxed state. This means that the accommodating fluidic IOL can be made to possess different base power in addition to a wide range of tuning. The base power and the tuning range define the functional range of the fluidic IOL. Eye surgeons can select the particular fluidic IOL according to these two parameters to best match the need for individual patients. Considering the depth of focus and the diversity of eye structure among patients, fluidic IOLs can be fabricated with different base powers having an increment of 0.5 D.

Several methods may be implemented to set the base optical power of a fluidic IOL. An easy and precise method involves controlling the amount of fluid introduced to the lens chamber and the reservoir. A fluidic IOL may contain approximately 100 uL (0.1 cc) of optical fluid. Using a syringe, the amount of fluid can be controlled by 0.5 uL or less. To achieve a power increment of 0.5 D for a device with a tuning range of 10 D, the required accuracy for the amount of lens fluid may be over 1 uL, which can be achieved with fluid injection devices that may be readily available. Another method to alter the base power of a fluidic IOL may be to alter the outer diameter or the shape of the reservoir. As stated before, the presence of the ring can decouple the geometry of the lens from the reservoir, therefore varying the geometry of the reservoir, which can change the optical power without compromising the optical quality of the lens.

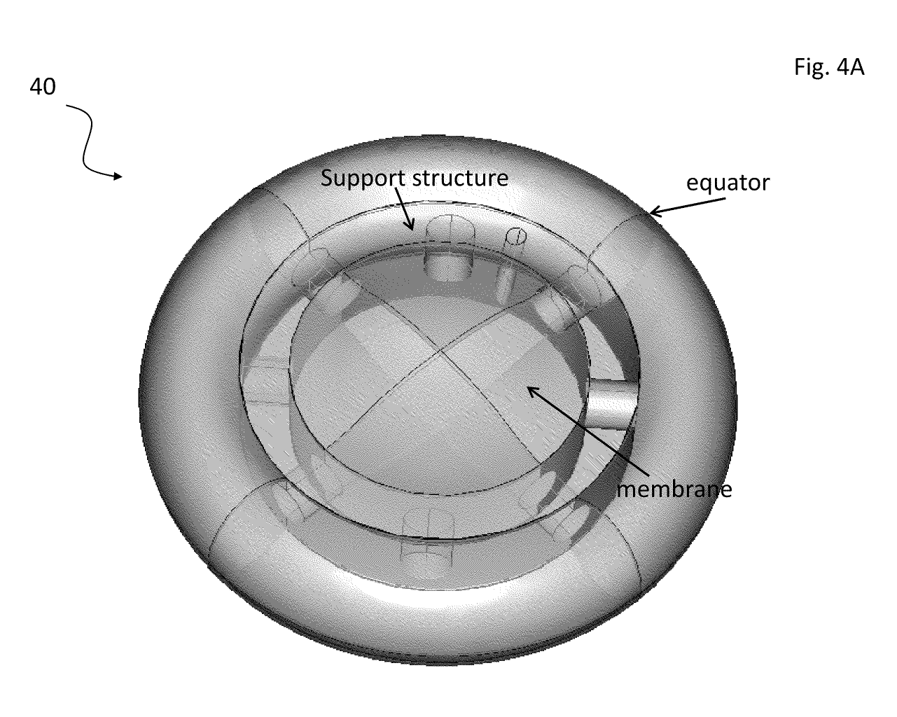

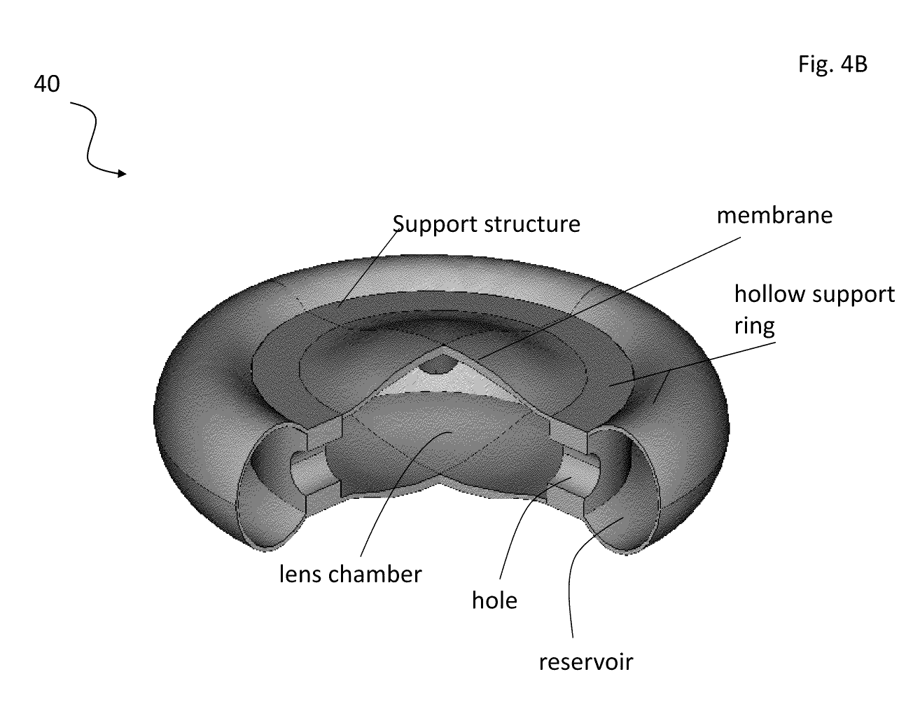

FIG. 4 describes another embodiment of accommodating fluidic IOL in accordance with the present invention. This device comprises three parts: a lens chamber, a support ring and a reservoir. Both the lens chamber and the outer reservoir may be filled with optical fluid. The fluid can move from one chamber to the other through the holes on the wall of the support ring. The outer diameter of the fluidic IOL may be slightly larger than the inner equatorial diameter of the capsular bag so that after implant, the IOL may be fastened by the radial compression.

The outer reservoir chamber may be made of one continuous piece of membrane. When filled with optical fluid, the membrane of the reservoir bulges up to form a donut shape around the lens chamber. The membrane of the reservoir may be thicker than the membrane of the lens chamber, making it more difficult to be stretched. Due to the higher stiffness of the reservoir membrane, the compression expels optical fluid into the lens chamber to cause the curvature change of the lens, instead of causing the bulge of the donut-shaped reservoir.

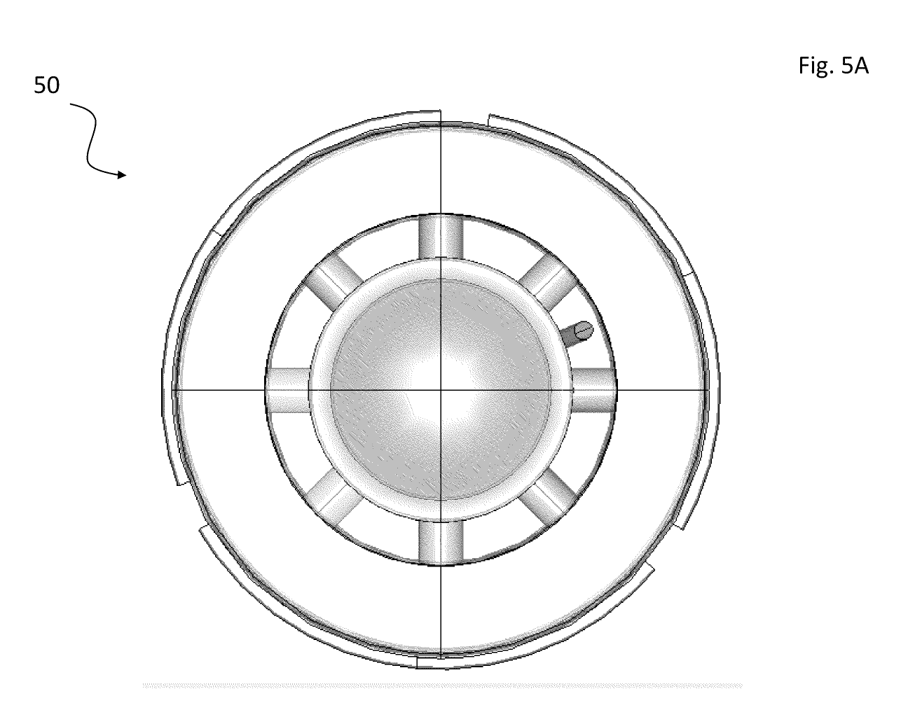

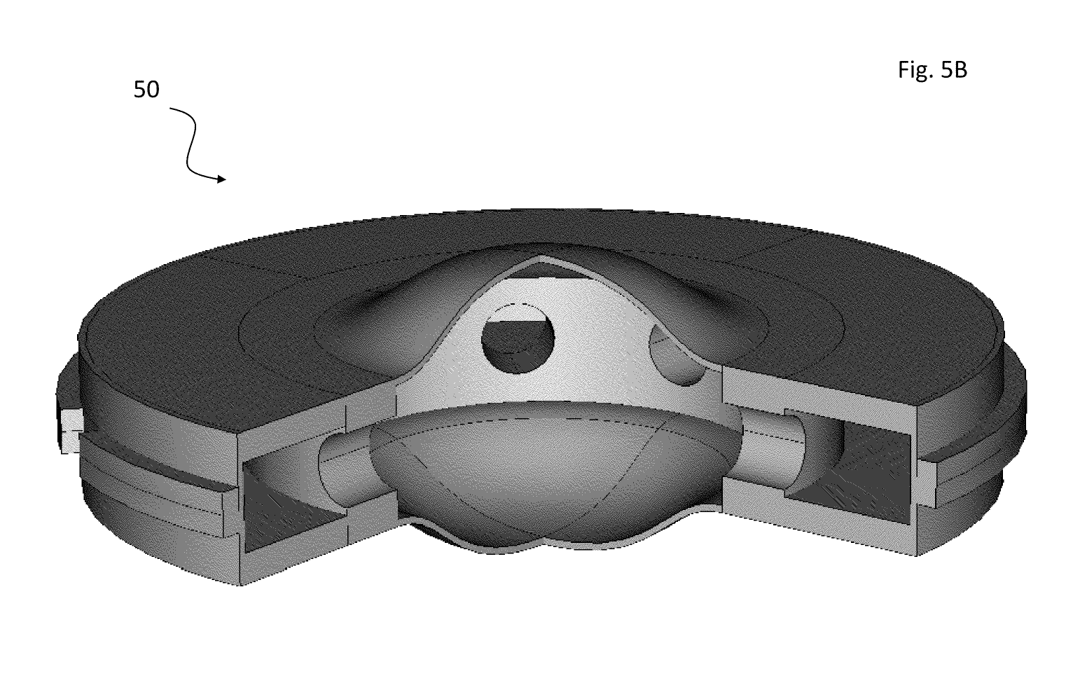

FIG. 5 shows another embodiment of accommodating fluidic IOL in accordance with the present invention. Both the lens chamber and the outer reservoir may be filled up with optical fluid. There may be any number of holes (e.g., 8) on the side wall of the support ring, through which fluid can move from one chamber to another in accordance with the deformation of a capsular bag. The outer diameter of the fluidic IOL may be slightly larger than the capsular bag so that after implantation, the device may be fastened by the radial tension.

The outer reservoir may have a thick anterior and posterior surface but a thin equatorial wall. There may be any number (e.g., 3) of relatively rigid arc elements attached to the flexible thin equatorial membrane. The deformation of the capsular bag may be transformed into the movement of the arc elements outside the reservoir. The width of the rigid arc elements may be slightly smaller than the width of the thin equatorial membrane so that these arc elements can push the thin reservoir wall inward to cause fluid flow from the reservoir to the lens chamber. Between two arc elements there may be a space. This space avoids end-to-end contacts of neighboring arc elements when the equatorial diameter of the capsular bag shrinks during accommodation. Since the anterior and posterior surfaces of the reservoir may be more rigid than the membranes of the lens chamber, optical fluid may be mostly pushed into the lens chamber when the arc elements move inward.

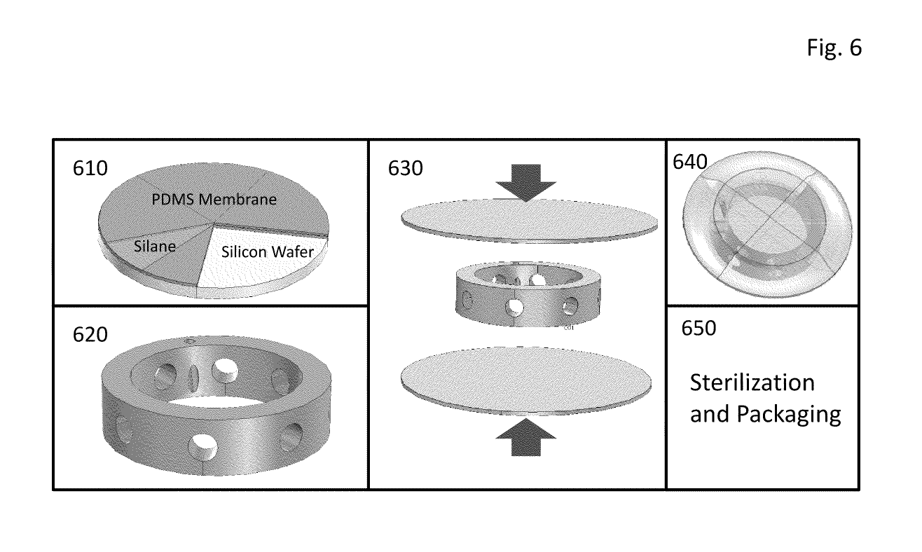

A method of preparing a fluidic IOL can be divided into five major steps, as shown in FIG. 6: membrane preparation 610, support ring preparation 620, bonding 630, filling and sealing 640, sterilization and packaging 650.

Membrane Preparation 610

Considering the limited force that can be provided by cilliary muscle, a thin membrane may be required. In order to make a smooth membrane with optical quality, uncured PDMS may be spun onto a silicon wafer. By controlling the spin speed, desired thickness can be achieved. The typical speed of spinning of PDMS may be 1500 rpm for 30 seconds. The material may be then cured in a 50 C oven for 1 hour. In the case of thin membrane, however, electrostatic effects can make the membrane crumple, make the membrane difficult to handle after the membrane may be peeled off from the substrate. To avoid such a problem, a much thicker (e.g. 1 mm) PDMS handle layer may be often formed on a silicon wafer first before the spin coating of the thin membrane. Between This handling layer and the thin membrane, a silane coating may be applied to make the interface more inert, which facilitates the separation of these two layers later on.

Support Ring Preparation 620

The support ring may be a critical component for the performance of a fluidic IOL. The circularity of the ring directly affects the optical quality of the optics. A ring deviating from a perfect circle or a sufficiently near-perfect circle can introduce astigmatism, degrading the image quality. In addition, the support ring may need to be soft and elastic enough to have the fluidic IOL pass through a small incision via a lens dispenser. On the other hand, the ring may have to be significantly more rigid than the lens membrane to effectively isolate the lens region from any non-uniformity and geometric imperfections of the capsular bag.

In order to make a good ring, a mold may be machined. The mold can be separated into two parts to facilitate demolding. First, PDMS may be mixed at a ratio of 1:10 and poured onto the mold. Then the mold may be placed in a desiccator and vacuumed to get rid of any trapped gas bubbles. After curing, the support ring may be demolded. Holes on the side wall of the ring may be punched by needles; and a fluid inject channel between the top surface and the side wall of the ring may be formed in a similar method.

Bonding 630

A most desirable method for joining two materials together includes bonding without any intermediate joining material to provide enough bonding strength. PDMS/PDMS bonding can be achieved without a joining layer if the PDMS surface may be activated first. Two thin membranes and the support ring may be treated with UV-ozone for surface activation. A uniform pressure may be applied to two membranes that sandwich the support ring. Keeping the applied pressure, the membranes and the sandwiched ring structure may be placed in an oven for a period of time (e.g., more than 8 hours) at a temperature (e.g., 90 degrees Celsius). Over This period, each membrane may be bonded to the respective surface of the support ring, and the two membranes may be bonded in the peripheral where the membranes may be in contact. After the bonding of all three pieces may be completed, a structure with a central lens chamber and a reservoir divided by the support ring with through holes on the side wall is achieved. At This stage, the handle layers that have been attached to the PDMS membranes to provide mechanical support and prevent membrane crumpling may be peeled off from two thin membranes.

Filling and Sealing 640

The fluidic IOL may need to be filled up with a precise amount of optical fluid without bubbles. A fluid supply reservoir may be connected to the fill port of the device via a needle injector. The entire setup may be placed in a vacuum chamber until the right amount of fluid may be injected and there are no gas bubbles left in the device. Fluid filling in vacuum can expand a trapped bubble to about 1000 times its size in atmosphere, making the bubble identifiable. Finally, the fluid-fill channel may be plugged to seal.

Sterilization and Packaging 650

After fabrication, the device may be sterilized and packaged following the standard procedure for devices of the same class, as is known in the art.

As an implant device, the fluidic IOL should not degrade or decompose over patient's lifetime. In particular, the fluidic IOL material selected from the fluidic IOL should not degenerate under a long period of UV exposure. Since light having wavelength shorter than 285 nm cannot reach the surface of the earth and light having wavelength between 285 and 300 nm may be strongly absorbed by the cornea, the UV light reaching the fluidic IOL has a wavelength primarily between 300 and 400 nm. It may be important that neither the lens body nor the optical fluid changes its mechanical or optical properties under such UV exposure.

Although the UV light in the 300-400 nm spectral range should not alter the properties of the lens material and the fluid, it may be desirable that the fluid/gel can absorb such UV light to prevent the light from reaching the retina. This will protect retina from lesions caused by UV irradiation. To prevent the deleterious short-wavelength radiation after cataract surgery, fluid/gel may have low UV transmission but high transmission of visible light.

The density of fluid or gel also may need to be taken into account. If the profile of fluidic IOL may be distorted by the weight of fluid due to the effect of gravity, additional astigmatism, coma, and spherical aberration occur and the image quality could be compromised. Furthermore, a fluidic IOL that may be either too heavy or too light may result in lens movement away from the optical center of the eye and surgical failure. To minimize the gravity effect, the density of fluid can be as close to the density of aqueous humor as possible.

Membranes for a fluidic IOL may need to be thin and easily stretchable, impermeable to fluid and/or gel, and as smooth as an optical-quality surface (i.e. a surface roughness of no more than 3 nm). PDMS (e.g. sylgard 184 from Dow Corning or Gelest 1.41) satisfies all the above requirements except that it may be impermeable to only a limited number of fluids (e.g. polyphenyl ether, SL5257 from Nusil). In some cases, although the effect of permeation may be not obvious, fluid may still be incorporated into the membrane to cause “swelling”. Either swelling or permeation could render failures of fluidic IOL as an implanted accommodating lens. A method of preventing swelling and fluid permeation may be to employ a multi-layer membrane structure. For example, the first layer may contain a melt-processible thermoplastic such as thermoplastic elastomer. The second layer may contain a second melt-processible thermoplastic chemically dissimilar to the melt-processible thermoplastic employed in the first layer. One layer of material may be responsible for prevention of fluid permeation, and the other layer of material produces the desired mechanical properties.

The publication by S. K. Thanawala and M. K. Chaudhury, “Surface modification of silicone elastomer using perfluorinated ether,” Langmuir, 16(3), 1250 (2000), describes how the surface tension of a PDMS film was lowered from 22 to 8 dyne/cm (mN/m) by mixing with 1 to 1.5% of a perfluorinated polyester. The final surface tension of 8 dyne/cm may be nearly equivalent to that of poly-tetrafluoroethylene or PTFE Teflon and would cause oils such as thioether or petroleum distillates to “bead” on its surface. In other words, these liquids should have a high contact angle (>85°) on the surface of perfluorinated polyether modified PDMS and may thus be repelled from it without permeating through it.

PDMS could be Dow Corning's Sylgard 184, Gelest 1.41, or Gelest 1.43. The fluorinated material can be derived from a DuPont product whose trade name may be Krytox, a perfluorinated polyester. The material reacts with the PDMS so that it may be chemically bonded to the elastomer and thus cannot be solvent-extracted or evaporate away. It may be added to the PDMS immediately after mixing both parts of the liquid elastomer kit and would thus participate in the cure. Curing conditions may vary. For example, they may be around 3 hours at 75° C. The viscosity of the Krytox may be low enough and the cure time is long enough that it migrates to the surface and preferentially puts fluorine on the surface and not in the bulk. This promotes lower surface tension at very small concentrations of the Krytox, and does not significantly affect the physical properties of the elastomer.

Dow Corning and Daikin developed a Fluoropolymer/Silicone Hybrid Coating to provide oil resistance (http://www.dowcorning.com/content/publishedlit/Easy-Clean-Stay-Clean.pdf). The hybrid material consists of a soluble fluoropolymer base, an isocyanate crosslinking compound and a silicone release agent. The combination of fluoropolymer and silicone gives superior release properties associated with low surface energy (hexadecane contact angel=34°). The Fluoropolymer/Silicone Hybrid Coating may be applied to the membrane forming a fluid permeation barrier.

Parylene coating on PDMS or other transparent elastomer may be another attractive approach for an impermeable membrane. Parylene coatings may be biocompatible and biostable and used in a wide range of medical and consumer applications. They provide excellent moisture, chemical and dielectric barrier protection. The coatings also have a low coefficient of friction for applications where lubricity may be a concern.

Parylene may be applied at room temperature with deposition equipment that controls the coating rate and ultimate thickness. Polymer deposition can take place at the molecular level. Raw material Dimer may be vaporized under vacuum and heated to a dimeric gas. The gas may be then pyrolized to cleave the dimer to its monomeric form. In the room temperature deposition chamber, the monomer gas can deposit as a transparent polymer film. The required thickness of a coating can vary based on the application, but thickness can range from the hundreds of angstroms to several mils, with the typical coating being in the microns range. The resultant coating provides an excellent barrier to fluid permeation to the lens fluid.

Coatings may be available in several formulations beginning with Parylene N that may be a carbon-hydrogen molecule, poly (para-xylylene), a completely linear, highly crystalline structure. Parylene C may be a carbon-hydrogen molecule with a chlorine atom on the benzene ring. Parylene D may be produced from the same dimer as Parylene N, modified by substitution of the chlorine atoms for two of the aromatic hydrogens. Parylene HT replaces the alpha hydrogen atom of the Parylene dimmer with fluorine.

Fluidic IOLs can be implanted following various procedures, including the procedure below:

STEP 1: Measure the shape of the capsular bag, including the thickness and the equatorial diameter of the bag, as well as its shape change during the process of accommodation.

The measured shape change of the capsular bag during accommodation can provide a reference for the expected surgical outcome, like the tuning range of fluidic intraocular lens, the far point of focus, and the near point of focus.

STEP 2: Determine the base power of the fluidic IOL.

The base power of IOL may be determined by patient's far-point focal length. The needed base power of the fluidic IOL may be calculated from the measurements of the corneal curvature, axial length of the eye, and anterior chamber depth.

STEP 3: Implant the fluidic IOL using known surgical protocols.

Surgeons prefer small incisions because wide incisions require longer recovery time, increase astigmatic complications, increase chances of collapse of the intraocular chambers during operation, and raise the risk of post-operative complications. To operate with a small incision, the accommodating fluidic IOL may need to be folded to have a minimum cross section for insertion.

The protocol of the implantation may vary. One protocol, a capsulotomy, includes removing the crystalline lens through an incision at the anterior capsule, and killing or removing remaining epithelial cells to inhibit further cell growth. To facilitate implantation of the fluidic IOL through the small incision, the fluidic IOL may be first inserted into an injector or dispenser. The fluidic IOL may be then pushed out of the injector or dispenser into patient's capsular bag. The IOL may be released in the capsular bag and adjusted and aligned to the proper position.

STEP 4: Determine the refractive value of the eye after implantation.

After implantation, the refractive value of the eye may be measured. If any data were incorrectly measured or calculated in the previous steps, the effect may be represented as a wrong refractive value of the eye.

STEP 5: Compare the refractive values of steps (b) and (d).

STEP 6: Change a fluidic IOL if necessary.

If the measured refractive power falls outside the acceptable range (e.g. 1 D), surgeons need to explant the previous fluidic IOL and implant another fluidic IOL of the correct base power.

The exemplary systems and methods of the invention have been described above with respect to particular systems and methods. One of skill in the art will appreciate alternative embodiments. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

We claim:

1. A method of manufacturing a fluidic intraocular lens device, the method comprising:
preparing a first optical membrane;
preparing a hollow support ring;
bonding the first optical membrane and the hollow support ring; and
filling at least the hollow supporting ring with fluid.

2. The method of claim 1, wherein the preparing a first optical membrane comprises:
spinning PDMS into a silicon wafer at a speed;
creating, based on the spinning, the first optical membrane; and
controlling the speed to achieve a desired thinness of the first optical membrane.

3. The method of claim 2, wherein the preparing a first optical membrane further comprises:
applying a silane coating to the silicon wafer; and
forming a PDMS handle layer on the silane-coated silicon wafer.

4. The method of claim 3, wherein the preparing a first optical membrane further comprises:
peeling off the PDMS handle layer from the silicon wafer.

5. The method of claim 1, wherein the one or more walls of the hollow support ring are at least partially elastic to enable the support ring to be folded or bent.

6. The method of claim 1, wherein the preparing a hollow support ring comprises:
pouring PDMS into a mold to create a molded PDMS form;
placing the molded PDMS form in a desiccator;
vacuuming the molded PDMS form to eliminate one or more gas bubbles from the molded PDMS form;
curing the molded PDMS form to create a first cured PDMS object; and
removing the first cured PDMS object.

7. The method of claim 6, wherein the preparing a hollow support ring further comprises:
creating one or more holes in one or more areas of the first cured PDMS object.

8. The method of claim 1, wherein the preparing a hollow support ring comprises:
creating a second cured PDMS object; and
bonding the first cured PDMS object and the second cured PDMS object to each other to form the hollow support ring.

9. The method of claim 8, wherein the bonding to form the hollow support ring comprises:
applying a UV ozone treatment to one or more surface areas of the first and second cured PDMS objects; and
joining the first cured PDMS object and the second cured PDMS object to create at least part of the hollow support ring.

10. The method of claim 1, wherein the hollow area inside the hollow support ring forms a reservoir, and wherein the bonding comprises:
bonding a second optical membrane and the hollow support ring, wherein the bonded first optical membrane, the hollow support ring and the second optical membrane form a lens chamber, wherein one or more surface areas of the hollow support ring include one or more holes that allow fluid to pass between the reservoir and the lens chamber.

11. The method of claim 1, wherein the filling comprises:
filling the reservoir and the lens chamber with the fluid.

12. A method of implanting a fluidic intraocular lens into an eye, the method comprising:
inserting the fluidic intraocular lens into the eye.

13. The method of claim 12, the method further comprising:
determining the shape of a capsular bag in the eye.

14. The method of claim 13, wherein the determining comprises:
determining an equatorial diameter and a thickness of the capsular bag; and
determining one or more surgical characteristics of the capsular bag, including one or more of a tuning range characteristic and a focus characteristic.

15. The method of claim 12, the method further comprising:
determining a base power of the fluidic intraocular lens.

16. The method of claim 15, wherein the determining comprises:
determining one or more of a cornea curvature of the eye, an axial length of the eye, an anterior chamber depth of the eye, or a focal length of the eye.

17. The method of claim 12, wherein the fluidic intraocular lens is in a folded or a bent state.

18. The method claim 12, the inserting comprises:
removing a crystalline lens from the eye;
inserting the fluidic intraocular lens into the eye; and
setting the fluidic intraocular lens at a desired position in the eye.

19. The method of claim 12, the method further comprising:
determining, after the inserting, a refractive value of the eye; and
removing, based on the determining, the fluidic intraocular lens from the eye; and
inserting a second intraocular lens into the eye.

20. An intraocular apparatus for implantation in an eye, said apparatus comprising:
a partially elastic ring structure, wherein at least part of the ring structure forms a reservoir;
a first lens membrane;
a second lens membrane, wherein the ring structure, the first lens membrane and the second lens membrane form a lens chamber;
one or more pathways between the reservoir and the lens chamber to enable exchange of fluid between the lens chamber and the reservoir, wherein the fluid is at least partially transparent to visible light and has a refractive index greater than 1.33; and
a pressure mechanism to create a force that causes the fluid in the reservoir to enter the lens chamber, thereby causing a shape of the first lens membrane to change.

21. The apparatus of claim 20, wherein the said membrane is made of Polydimethylsiloxane (PDMS), wherein the fluid is an optical gel or polyphenyl ether and has a density no greater than 1.25 grams/cm3, and wherein a circular portion of the lens chamber measures at least 5 mm in diameter.
